(19) United States
(12) Patent Application Publication
(10) Pub. No.: US 2010/0005707 A1
(43) Pub. Date: Jan. 14, 2010

(54) LOW-SOOT DIESEL FUELS COMPRISING A FUEL ADDITIVE, USE THEREOF AND THE USE OF THE FUEL ADDITIVE FOR PRODUCING LOW-SOOT DIESEL FUELS

(76) Inventor: Eberhard Jacob, Krailling (DE)

Correspondence Address: FAY SHARPE LLP, 1228 Euclid Avenue, 5th Floor, The Halle Building, Cleveland, OH, 44115, US

(21) Appl. No.: 12/499,930

(22) Filed: Jul. 9, 2009

(30) Foreign Application Priority Data
Jul. 9, 2008 (DE) 10 2008 032 254.7

(51) Int. Cl. C10L 1/18; C07C 43/04

(52) U.S. Cl. 44/385; 568/672; 44/443

(57) ABSTRACT

The invention relates to a low-soot diesel fuel comprising a fuel additive, to the uses thereof and to the use of the fuel additive for producing low-soot diesel fuels. The diesel fuels are mineral-based, optionally with additions of FAME or XTL diesel fuels, which are formulated with polyoxaalkanes of the general formula (I):

R¹(—O—CH₂—CHR²)ₘ—O—R³ (I)

In the formula (I), the R¹ radicals are each a straight-chain or branched alkyl radical, R² is a straight-chain or branched alkyl radical or H, and R³ is likewise a straight-chain or branched alkyl radical. In addition, m is ≧1, and the fuel additive is essentially free of toxic constituents. Diesel fuels formulated in this way, even at a low polyoxaalkane content, have significantly reduced soot formation. The proportion of premixed combustion and the density are increased, and the volumetric reduction in calorific values which occurs when CH₂ groups in long-chain alkanes are replaced by O groups can be compensated for. The invention further relates to a process for homogenizing diesel fuel/alkanol mixtures, in which addition of the polyoxaalkanes described affords a monophasic diesel fuel. Such mixtures are of interest with regard to falling crude oil reserves, because primary alcohols such as ethanol can be prepared readily and inexpensively from organic starting materials.

The invention relates to diesel fuels which burn with a low soot content and comprise at least one fuel additive. The invention further relates to the use of fuel additives for producing such diesel fuels and to a process for reducing soot formation in diesel engines and to a process for homogenizing diesel fuel/alkanol mixtures.

Owing to the great rise in demand for oil in the last few years and the associated significant price rise, synthetic fuels and fuels from renewable raw materials have gained ever greater significance. Such fuels generally comprise either hydrocarbons, which are prepared, for example, with the aid of the Fischer-Tropsch synthesis from raw materials such as gas, coal and plant residues, or from alcohols, which can be obtained, for example, from sugarcane by fermentation. Hydrocarbon fuels are generally produced with the aid of XTL technology, X being a placeholder for gas (G), coal (C) or biomass (B). The letters T and L in this context mean that the starting material is converted to a liquid. XTL technology is frequently based on Fischer-Tropsch synthesis at low temperature and affords a liquid fuel as the product.

XTL diesel fuels produced synthetically in this way lead, compared to mineral oil-based fuels, to combustion with an about 20% lower soot content (J. Krahl et al., 5th Internat. Colloquium Fuels, Jan. 12-13, 2005, Edt.: W. J. Bartz, TAE Ostfildern, p. 207/212). Such lower-soot combustion is desirable especially in city traffic and is currently being, and in the near future will be, legally limited by the crucial emissions standards, for example in the case of utility vehicle engines according to EU V (EU Directive 2006/51/EC) and EU VI (EU Directive will be published in 2009). Analogous legislation exists in the USA and Japan. Moreover, such diesel fuels allow the AGR rates (exhaust gas recycling) of the diesel engines to be increased significantly, in order thus to lower the NOₓ emissions without a rise in the soot emission and the fuel consumption.

Owing to the production technique via Fischer-Tropsch synthesis, XTL fuels comprise almost exclusively aromatics-free alkanes, whose density at 0.735 g/ml (manufacturer: Shell) and 0.765 g/ml (manufacturer: Sasol) is, however, 7.6 to 13% lower than the standard stipulates for diesel fuels (DIN EN 590, 3.2004 edition, requires a density of 0.820 to 0.845 g/ml). The initial boiling point of such a fuel is approx. 170° C., the final boiling point approx. 330° C. A disadvantage in the production of such fuels is likewise that the Fischer-Tropsch synthesis predominantly forms a crude product composed of straight-chain n-alkanes with up to 60 carbon atoms, which have to be converted in a downstream cracking and isomerization step partially to short-chain n-alkanes and isoalkanes. This further step is required because long-chain n-alkanes generally have high melting points, which adversely affects the flowability of the fuels, and can lead to blockage of the fuel filters in the event of cold temperatures, as, for example, in winter. The addition of isoalkanes to the fraction of n-alkanes allows filterability to be ensured even under cold conditions (cold filter plugging point, CFPP to DIN EN 590 in winter: −20° C.), because isoalkanes generally have lower melting points and thus improve the flowability of the mixture. In addition to the necessary isomerization step, this method, however, has yet a further disadvantage. The addition of isoalkanes to the n-alkanes also leads to a lowering of the cetane number of the fuel, such that the ignitability of the Fischer-Tropsch fuel is likewise reduced.

Even by means of XTL diesel fuels, soot evolution can be halved at best. As an alternative to the use thereof, it has already been proven repeatedly that a significant reduction in soot formation can also be achieved by means of oxygen-containing fuel additives. Such additives have the advantage that they lead to a lowering of the mean combustion temperature in the cylinder and increase the proportion of premixed combustion. Premixed combustion is understood to mean the combustion of a homogenized mixture of evaporated fuel and air, in contrast to diffusion combustion (inhomogeneous combustion of fuel droplets with soot formation). The oxygenates tested and used as fuel additives to date have predominantly been those in which the oxidation number of the carbon atoms which contain bound oxygen atoms is greater than +1. In particular, carbonates, e.g. dimethyl carbonate (DMC), and esters, e.g. dibutyl maleate (DBM) and fatty acid methyl esters (FAME), which contain carbonyl groups, have been used. In addition, acetals and polyacetals, e.g. butylal and methylal with a —C—O—C—O—C— base skeleton in which the central carbon atom is bonded to two adjacent oxygen atoms have been used (A. Bertola, K. Boulouchos, SAE Paper 2001-01-2885). In these cases, the oxidation states of the oxygen-bearing carbon atom at ≧+2 are already very high, which leads to a corresponding reduction in the calorific value. In the case of esters with —C═O(—OR)— groups, the combustion thereof leads to decarboxylation with local CO₂ elimination, and so the oxygen present can make little contribution to the lowering of soot formation. The use of those fatty acid methyl esters (FAME to EN 14214:2003) which contain approx. 11% oxygen as a fuel leads to lowering of particulate emissions compared to diesel fuel of 40-60% (J. Krahl et al., 5th Internat. Colloquium Fuels, Jan. 12-13, 2005, Edt.: W. J. Bartz, TAE Ostfildern, p. 207/212).

In addition, dialkyl ethers and other monoethers having 2-24 carbon atoms (WO 1995/025153 and U.S. Pat. No. 5,520,710) have been described as fuel additives for diesel fuels, as have ethylene glycol dimethyl ether and diethylene glycol dimethyl ether (SAE Paper 2000-01-2886). The ether oxygenates described have, however, generally either only a low oxygen content at acceptable boiling points, or, in the case of a high oxygen content, a very low boiling point, for instance dimethyl ether or diethyl ether, which makes the use thereof in fuels problematic.

An additional factor is that many low molecular weight ethers have a marked tendency to form peroxides, and so their use in practice is not effective. The diethylene glycol dialkyl ethers described to date, especially Cetaner®, a mixture of 20% 1,2-dimethoxyethane and 80% diethylene glycol dimethyl ether, have a significant soot-lowering action. Both components of Cetaner® are, however, extremely toxic substances whose general use as a fuel or fuel additive cannot be approved. A further disadvantage is that both substances are relatively volatile, which is additionally problematic in connection with their toxicity.

Alcohols are likewise less suitable as fuels because they either have very low cetane numbers (such as ethanol or methanol), or else, given acceptable cetane numbers, already have excessively high melting points and only a low oxygen content. An additional factor in the case of the low molecular weight alcohols (1-4 carbon atoms) is that they have only limited miscibility, if any, with diesel fuels (Nylund et al. “Alcohols/Ethers as Oxygenates in Diesel Fuel”, Report TEC 3/2005).

It is therefore an aim of the invention to further develop the diesel fuels described at the outset in such a way that the disadvantages of known diesel fuels are substantially eliminated. At the same time, the diesel fuels should especially have reduced soot formation and lead to a lowering of the ignition temperature and hence of NOₓ formation, and be essentially free of toxic constituents. Moreover, the additives present in the diesel fuel for soot reduction in the course of combustion should have a maximum oxygen content. On the other hand, the oxygen content of the fuel should be kept just as low as the soot-lowering action requires, in order to enable a minimum volumetric calorific value loss compared to diesel fuel to DIN EN 590. The boiling points of the additives used should preferably be selected such that they meet the requirements of the international standards for diesel fuels (for example, according to DIN EN 590, only 5% of the components may boil at higher than 360° C.). Moreover, the diesel fuel should have good cold performance (CFPP according to test method EN 116 in DIN EN 590) and hence enable good filterability under cold conditions. The diesel fuel should preferably approach the density requirements of the standard EN 590. Finally, the ignition properties of the diesel fuels should remain at a high level (cetane number>50). The flashpoint should be <55° C. as in the case of diesel fuel.

According to the invention, this aim is addressed by providing a diesel fuel comprising a fuel additive comprising at least one polyoxaalkane of the general formula (I):

R¹(—O—CH₂—CHR²)ₘ—O—R³ (I),

where R¹ is a straight-chain or branched alkyl radical, R² is a straight-chain or branched alkyl radical or H, preferably a straight-chain alkyl radical, especially a methyl radical, and more preferably H, R³ is a straight-chain or branched alkyl radical, preferably a straight-chain alkyl radical, m is ≧1, and wherein the fuel additive is essentially free of toxic constituents.

The term “at least one polyoxaalkane” explicitly includes mixtures of polyoxaalkanes.

“Toxic” is understood to mean toxic according to the German Dangerous Substances Act (GefstoffV 23.12.2004, BGBI. I p. 3758). For example, the inventive diesel fuel does not contain any polyethylene glycol dimethyl ether of the formula CH₃O(C₂H₄O)ₙCH₃ where n=1-3. Toxic constituents shall also include teratogenic constituents.

The inventive diesel fuels have significantly reduced soot formation.

In addition, the fuel addition in conjunction with a diesel fuel, especially a hydrogenated vegetable oil, preferably NexBTL, or an XTL diesel fuel, leads to an increase in the density and hence to a compensation for the volumetric calorific value reduction which occurs on replacement of CH₂ groups in long-chain alkanes by —O— groups.

In addition, the air required by the engine is lowered by up to one third. This has a series of advantages: the cylinder charge (quotient of charge pressure and air pressure) can be reduced by up to one third compared to engine operation with XTL fuel. The expenditure for the compression of the charge air falls correspondingly, which leads to the effect that an expensive two-stage compression can be dispensed with and a cheaper one-stage charging group can be used. Moreover, the amount of exhaust gas produced is likewise approx. one third lower when the air required for diesel engine combustion is one third lower. The catalytic exhaust gas aftertreatment then saves one third of catalyst volume and can be intensified and simplified.

The polyoxaalkanes derive from alkanes in which a plurality of CH₂ groups have been replaced by oxygen atoms.

The R¹ radical preferably denotes an alkyl radical with a chain length of 1-4 carbon atoms, more preferably a straight-chain alkyl radical, since these cause the cetane numbers of the oxygenates to rise. Methyl, ethyl and n-butyl radicals can also be produced biogenically. This is one means of upgrading the bioalcohols methanol, ethanol and n-butanol, since their direct use as diesel fuels is very difficult especially as a result of their low ignitability. The cetane numbers of methanol, ethanol and n-butanol are 3, 8 and 17 respectively.

The R² radical preferably denotes a straight-chain or branched alkyl radical having 1-4 carbon atoms or H, more preferably a straight-chain alkyl radical or H. The R² radical contains more preferably 0 to 2 carbon atoms and most preferably 0 to 1 carbon atom. When the R² radical does not contain any carbon atoms, R²═H.

For reasons of cost, R₂ is preferably H.

The R³ radical likewise preferably denotes a straight-chain or branched alkyl radical having 1-4 carbon atoms, more preferably a straight-chain alkyl radical or H.

In a further preferred embodiment, R¹ and R³ are each a methyl, ethyl and/or butyl radical. The use of such compounds leads to improved solubility of the polyoxaalkane in the diesel fuel, i.e. the miscibility is increased.

m is, as mentioned, ≧1, preferably ≧4, especially 4-16 and more preferably 1 to 12. Such compounds are especially inexpensive.

The polyoxaalkane is preferably free of carbon atoms with an oxidation number of >+1, more preferably free of carbon atoms with an oxidation number of >0.

In a preferred embodiment, the at least one polyoxaalkane is a polyalkylene glycol diethyl ether, especially a polyethylene glycol dimethyl ether of the general formula CH₃O(C₂H₄O)ₘCH₃ where m=4 or a mixture of m=4 to m=12, preferably of m=4 to m=8. It is preferably tetraethylene glycol dimethyl ethyl.

In a further preferred embodiment, the polyalkylene glycol dialkyl ether is selected from the group consisting of ethylene glycol methyl butyl ether, ethylene glycol ethyl butyl ether, ethylene glycol dibutyl ether, diethoxypropane, diethyoxybutane, ethoxymethoxypropane, diethylene glycol ethyl methyl ether, diethylene glycol diethyl ether, diethylene glycol butyl ethyl ether, diethylene glycol dibutyl ether, triethylene glycol ethyl methyl ether, triethylene glycol diethyl ether, triethylene glycol butyl methyl ether, triethylene glycol butyl ethyl ether, tetraethylene glycol ethyl methyl ether, tetraethylene glycol butyl methyl ether, dipropylene glycol dimethyl ether and polypropylene glycol dimethyl ether having 3-5 propylene units.

The polyoxaalkane is more preferably a polyethylene glycol dibutyl ether, especially a polyethylene glycol dibutyl ether with a mean molecular weight of about 300, such as Polyglykol BB 300 (obtainable from Clariant Produkte GmbH). With this polyoxaalkane, it is possible to formulate diesel fuels which, with a correspondingly high content, meet the density requirements of DIN EN 590.

The boiling point of the polyoxaalkane is preferably between about 100° C. and about 450° C., more preferably between about 150 and about 375° C. and most preferably between about 170 and about 330° C. When the boiling point of the polyoxaalkane is equal to or less than approx. 330° C., this increases the proportion of the premixed, low-soot combustion and simultaneously lowers the proportion of soot-forming diffusion combustion of the unevaporable fuel droplets.

In a further preferred embodiment, the polyoxaalkane has a melting point of less than about −10° C., preferably of less than about −20° C. This has the advantage that the CFPP required in the standard DIN EN 590 can be achieved in the case of mixing with n-alkanes (high melting point).

In a further preferred embodiment, the polyoxaalkane has a density of about 0.8 to about 1.1 g/ml, preferably of about 0.85 to about 1.0 g/ml. A high density has the advantage that this allows the possibly lower density of a diesel fuel to be compensated for, such that the inventive diesel fuel satisfies the EN 590 standard or approaches this standard.

In a further preferred embodiment, the flash point of the polyoxaalkane is greater than about 55° C. This has the advantage that DIN EN 590 is satisfied.

The starting substance used for the inventive diesel fuel may be any diesel fuel. However, the diesel fuel used is preferably essentially free of aromatic constituents, since such constituents are generally responsible for enhanced soot formation. The diesel fuels used are more preferably hydrogenated vegetable oil, especially NexBTL, or XTL diesel fuels. XTL as used here shall include hydrocarbons which have been prepared via a gas to liquid (GTL), coal to liquid (CTL) or biomass to liquid (BTL) process. Most preferred is the use of GTL diesel fuel.

The diesel fuel used preferably has a density of about 0.7 to 0.8 g/ml, in order not to allow the density to fall too greatly below the standardized value of 0.82 g/ml, and hence the volumetric calorific value also falls only slightly.

The inventive diesel fuels, i.e. those containing fuel additive, preferably have a density of about 0.8 to about 0.845 g/ml, in order to almost satisfy the standard (EN 590).

The inventive diesel fuel is preferably essentially free of components with a boiling point of more than about 450° C., in order to satisfy the diesel fuel standards with regard to boiling behaviour.

The inventive diesel fuel preferably contains less than 5% by volume of components with a boiling point of more than about 360° C. (EN 590).

In a further preferred embodiment, the proportion of the polyoxaalkane in the inventive diesel fuel is up to about 20% by volume, preferably up to about 10% by volume and more preferably up to about 5% by volume. The lower the proportion of the additive is, the less expensive is the inventive diesel fuel. In addition, the lower the proportion of the additive, the smaller the lowering of the calorific value of the inventive diesel fuel.

More particularly, the inventive diesel fuel contains about 5 to 20% by volume of fatty acid methyl ester (FAME). This has the advantage that the biogenic constituents of the diesel fuel are increased, which incidentally is legally stipulated in many countries. Furthermore, FAME has the property of serving as a solubilizer. This has the advantage that more polyoxaalkane of the general formula (I) can be brought into solution in the diesel fuel. The inventive diesel fuel preferably contains about 5 to 10% by volume of FAME. This has the advantage that it allows up to about 10% by volume of polyoxaalkane, especially polyethylene glycol dimethyl ether of the general formula CH₃O(C₂H₄O)ₘCH₃ where m=4 to m=8 to be brought into solution.

A further embodiment of the present invention relates to the use of a fuel additive for producing diesel fuels. The fuel additives used comprise at least one polyoxaalkane of the general formula (I):

R¹(—O—CH₂—CHR²)ₘ—O—R³ (I),

where R¹ is a straight-chain or branched alkyl radical, R² is a straight-chain or branched alkyl radical or H, R³ is a straight-chain or branched alkyl radical, m≧1, and wherein the fuel additive is essentially free of toxic constituents, wherein “toxic” should be understood as defined above.

For R₁, R₂, R₃ and m, the same definitions apply as mentioned above. The aforementioned preferred polyoxaalkanes, for example the polyalkylene glycol dialkyl ethers mentioned, are likewise preferred for use.

The polyoxaalkane used in accordance with the invention is preferably free of carbon atoms with an oxidation number of >+1, more preferably free of carbon atoms with an oxidation number of >0.

The polyoxaalkane is preferably a polyethylene glycol dibutyl ether. Through the use of this polyoxaalkane, it is possible to produce diesel fuels which satisfy the density requirements of EN 590.

Preference is likewise given to polyethylene glycol dimethyl ether, especially tetraethylene glycol dimethyl ether.

The boiling point of the polyoxaalkane used is between about 100° C. and about 450° C., preferably between about 150 and about 375° C. and more preferably between about 170 and about 330° C. When the boiling point of the polyoxaalkane is equal to or less than 300° C., this increases the proportion of premixed, low-soot combustion and simultaneously lowers the proportion of soot-forming diffusion combustion of the unevaporable fuel droplets.

In a further preferred embodiment, the polyoxaalkane has a melting point of less than about −1° C., preferably of less than about −20° C.

In a further preferred embodiment, the polyoxaalkane has a density of about 0.8 to about 1.1 g/ml, preferably of about 0.85 to about 1.0 g/ml. A high density has the advantage that use of such an additive allows a possibly lower density of a diesel fuel to be balanced out, such that the inventive diesel fuel satisfies the EN 590 standard.

In a further preferred embodiment, the flash point of the polyoxaalkane is greater than about 55° C. This has the advantage that DIN EN 590 is satisfied.

In a preferred embodiment, the polyoxaalkane is used to produce a diesel fuel which is essentially free of aromatic constituents. Especial preference is given to using the polyoxaalkane for mixing with hydrogenated vegetable oil, especially NexBTL, and with XTL diesel fuels, especially GTL diesel fuels.

In a preferred embodiment, the amount of the fuel additive is preferably selected such that the density of the resulting diesel fuel is from about 0.8 to about 0.845 g/ml.

The present invention also relates to a process for reducing soot formation in the course of combustion of diesel fuels, wherein polyoxaalkanes of the aforementioned general formula (I) are added to a diesel fuel, and wherein the same definitions of R¹, R², R³, m and the fuel additive as described above for diesel fuels shall apply. When the process is employed, it is especially also possible to use any of the aforementioned diesel fuels. The diesel fuel used is, however, preferably a fuel which is free of aromatic constituents, and more preferably a hydrogenated vegetable oil, especially NexBTL, or an XTL diesel fuel.

The present invention also relates to a process for homogenizing diesel fuel/alkanol mixtures, which comprises the addition of polyoxaalkanes of the general formula (I) as a solubilizer and cetane number improver, wherein the same restrictions for R¹, R² and R³, m and the fuel additive as described above for diesel fuels shall apply. When the process is employed, it is also possible to use any of the aforementioned diesel fuels.

“Homogenization”, as used here, describes the conversion of a polyphasic mixture to a mixture with only one phase. The alcohol used is preferably a primary alcohol, more preferably a primary alcohol having 1 to 6 carbon atoms and most preferably ethanol or n-butanol. Ethanol is an alternative fuel to mineral oil-based fuels, which can be produced inexpensively from organic starting materials by fermentation. n-butanol has the advantage that will be producible in the future by a biochemical process from cellulose and hence is not in competition with food production.

Diesel fuel/alkanol mixtures are of interest with regard to the increasing scarcity of mineral oil-based fuels, because especially ethanol can be obtained by fermentation from renewable raw materials. Such bioethanol can be produced very inexpensively in Brazil, for example, and, by addition to conventional diesel fuel, can increase the total amount of fuel.

However, diesel fuel/alkanol mixtures generally cannot be used as a fuel in the form of a two-component mixture, because the two components are miscible with one another only in minor proportions, if at all. This is especially true of the low molecular weight alkanols methanol and ethanol. However, through the addition of polyoxaalkanes of the general formula (I), it is possible to produce diesel fuel/alkanol mixtures which are monophasic at the use temperature and which simultaneously allow the addition of large amounts of alkanol to the diesel fuel. In such three-component mixtures of diesel fuel/alkanol and additive, the amount of additive added is preferably at least about 4%, more preferably at least about 5 to 11%. The amount of the alkanol component is generally about ≧10%, preferably about ≧20% and most preferably about ≧30%.

The invention is illustrated further hereinafter by examples. The examples shall, however, in no way limit or restrict the present invention.

### EXAMPLE 1

### Production of a Stable Solution of GTL Diesel Fuel and Oxygenate

The GTL fuel used was GTL from Sasol/FM/No. 243 with a density at 20° C. of 0.7656 g/cm³. The oxygen content in the end mixture was 11% by weight. The densities of the particular mixtures are reported in Table 1.

TABLE 1

| | | | | Density at |
|---|---|---|---|---|
| | Diesel fuel | Glyme | Oxygen | 20° C. |
| | (% m/m) | (% m/m) | (% m/m) | (g/cm³) |
| Diethylene glycol ethyl | 66.46 | 33.54 | 11.0 | 0.8071 |
| methyl ether | | | | |
| Diethylene glycol diethyl | 62.84 | 37.16 | 11.0 | 0.8083 |
| ether | | | | |
| Dipropylene glycol | 62.84 | 37.16 | 11.0 | 0.8078 |
| dimethyl ether | | | | |
| Tetraethylene glycol | 69.44 | 30.56 | Maximum solubility | |
| dimethyl ether | | | 6% | |
| Polyglykol BB 300 | 55.47 | 44.53 | 11.0 | 0.8253 |
| Diethylene glycol dibutyl | 50.00 | 50.00 | 11.0 | 0.8176 |
| ether | | | | |

| | | | |
|---|---|---|---|
| Polyglykol DME | 69.53 | 30.47 | Maximum solubility |
| 250/DME 500 (1:4) | | | 2% |

Polyglykol BB 300 is a polyethylene glycol dibutyl ether with a mean molecular weight of about 300 (manufacturer: Clariant Produkte GmbH, 84504 Burgkirchen).
Polyglykol DME 250 and DME 500 are polyethylene glycol dimethyl ethers with a mean molecular weight of, respectively, about 250 and 500 (manufacturer: Clariant Produkte GmbH, 84504 Burgkirchen).

It is clear from Table 1 that the addition of polyoxaalkanes to GTL diesel fuels makes it possible to produce diesel fuels which have a significantly higher density than pure GTL diesel fuels. In the case of Polyglykol BB 300, the diesel fuel/glyme mixture also satisfies the density criteria of the EN 590 standard. It was additionally found that tetraethylene glycol dimethyl ether and Polyglykol DME 250/DME 500 mixtures are not suitable for formulating diesel fuels with an oxygen content of 11%. Both additives have oxygen contents of about 36%, which leads to the effect that the miscibility with nonpolar GTL diesel fuel is lowered.

### EXAMPLE 2

On a MAN one-cylinder research engine with a swept volume of 1.75 l, an engine power of 55 kW, a common rail injection system (rail pressure 1800 bar), a compression of 20.5, a commencement of injection before the upper dead point of crank angle −8° and an AGR rate of 20%, a fuel mixture of 95% by volume of diesel fuel to EN 590 and 5% by volume of tetraethylene glycol dimethyl ether was tested.

The results can be taken from Table 2 below. The comparison used was pure diesel fuel to EN 590.

TABLE 2

| Operating point | BP 1 | BP 2 | BP 3 | BP 4 |
|---|---|---|---|---|
| Soot reduction (NOx | −41.2 | −53.1 | −52.9 | −27.4 |
| emission level constant) % | | | | |
| Speed rpm | 914 | 1542 | 1542 | 1800 |
| Torque Nm | 75 | 200 | 270 | 140 |
| Average pressure bar | 6.3 | 15.7 | 20.7 | 11.7 |
| Air ratio | 3.1 | 1.35 | 1.35 | 1.9 |

This test shows that the addition of tetraethylene glycol dimethyl ether leads to significant soot reduction and, the lower the air excess in the combustion (see air ratio in Table 2), the higher said soot reduction is.

### EXAMPLE 3

Several diesel fuel/ethanol mixtures with polyoxaalkanes as solubilizers were prepared. To this end, diesel/ethanol mixtures with a maximum ethanol content of ≧30% by weight were prepared. The diesel fuel used was GTL from Sasol/FM No. 243 with a density at 20° C. of 0.7656 g/cm³, and Aral Ultimate with a density at 20° of 0.8236 g/cm³ and ethanol with a density at 20° of 0.7893 g/cm³. The densities and the corresponding oxygen contents of the corresponding mixtures are reported in Table 3.

TABLE 3

| | | | | | | Density at |
|---|---|---|---|---|---|---|
| | | Diesel | Glyme | Ethanol | Oxygen | 20° C. |
| | | (% m/m) | (% m/m) | (% m/m) | (% m/m) | (g/cm³) |
| GTL diesel | Diethylene | 68.3 | 0.7 | 31.0 | immiscible | |
| | glycol dibutyl | 65.6 | 3.5 | 31.0 | immiscible | |
| | ether | 62.1 | 6.9 | 31.0 | 12.3 | 0.7776 |
| | Polyglykol | 65.6 | 3.5 | 31.0 | immiscible | |
| | BB 300 | 62.1 | 6.9 | 31.0 | 12.5 | 0.7797 |
| Aral | Diethylene | 65.5 | 3.5 | 31.0 | immiscible | |
| Ultimate | glycol dibutyl | 62.1 | 6.9 | 31.0 | 12.3 | 0.8147 |
| diesel | ether | | | | | |
| | Polyglykol BB | 65.6 | 3.5 | 31.0 | immiscible | |
| | 300 | 62.1 | 6.9 | 31.0 | 12.5 | 0.8170 |

| | | | | |
|---|---|---|---|---|
| — | 83.3 | — | 16.7 | immiscible |

It becomes clear from Table 3 that stable diesel fuel/ethanol mixtures can be produced when relatively small amounts of polyoxaalkane are used.

1. A diesel fuel comprising a fuel additive comprising at least one polyoxaalkane of the general formula (I):
R¹(—O—CH₂—CHR²)ₘ—O—R³ (I),
where R¹ is a straight-chain or branched alkyl radical, R² is a straight-chain or branched alkyl radical or H, R³ is a straight-chain or branched alkyl radical, m is ≧1, and wherein the fuel additive is essentially free of toxic constituents.

2. A diesel fuel according to claim 1, wherein the diesel fuel contains up to about 20% by volume, preferably up to about 10% by volume and more preferably up to about 5% by volume of polyoxaalkane.

3. A diesel fuel according to claim 1, wherein the diesel fuel contains about 5-20% by volume of fatty acid methyl esters (FAME).

4. A diesel fuel according to claim 1, wherein m≧4.

5. A diesel fuel according to claim 4, wherein R² is H.

6. A diesel fuel according to claim 1, wherein R¹ and R³ are each a methyl, ethyl and/or butyl radical.

7. A diesel fuel according to claim 1, wherein the diesel fuel is a hydrogenated vegetable oil, especially NexBTL, or an XTL diesel fuel.

8. A diesel fuel according to claim 1, wherein the polyoxaalkane is free of carbon atoms with an oxidation number of >+1.

9. A diesel fuel according to claim 1, wherein the at least one polyoxaalkane is a polyalkylene glycol dialkyl ether.

10. A diesel fuel according to claim 9, wherein the polyalkylene glycol dialkyl ether is a polyethylene glycol dimethyl ether of the general formula CH₃O(C₂H₄O)ₘCH₃ where m=4 or a mixture of m=4 to m=12.

11. A diesel fuel according to claim 10, wherein the polyethylene glycol dimethyl ether is tetraethylene glycol dimethyl ether.

12. A diesel fuel according to claim 9, wherein the polyalkylene glycol dialkyl ether is selected from the group consisting of ethylene glycol methyl butyl ether, ethylene glycol ethyl butyl ether, ethylene glycol dibutyl ether, diethoxypropane, diethyoxybutane, ethoxymethoxypropane, diethylene glycol ethyl methyl ether, diethylene glycol diethyl ether, diethylene glycol butyl ethyl ether, diethylene glycol dibutyl ether, triethylene glycol ethyl methyl ether, triethylene glycol diethyl ether, triethylene glycol butyl methyl ether, triethylene glycol butyl ethyl ether, tetraethylene glycol ethyl methyl ether, tetraethylene glycol butyl methyl ether, dipropylene glycol dimethyl ether and polypropylene glycol dimethyl ether having 3-5 propylene units.

13. A process for reducing soot formation in diesel engines, wherein a fuel additive comprising at least one polyoxaalkane of the general formula (I)
R¹(—O—CH₂—CHR²)ₘ—O—R³ (I),
where R¹ is a straight-chain or branched alkyl radical, R² is a straight-chain or branched alkyl radical or H, R³ is a straight-chain or branched alkyl radical, m is ≧1, and wherein the fuel additive is essentially free of toxic constituents, is added to a diesel fuel.

14. A process for homogenizing and improving the self-ignition capacity of diesel fuel/alkanol mixtures, comprising the addition of a fuel additive comprising at least one polyoxaalkane of the general formula (I)
R¹(—O—CH₂—CHR²)ₘ—O—R³ (I),
where R¹ is a straight-chain or branched alkyl radical, R² is a straight-chain or branched alkyl radical or H, R³ is a straight-chain or branched alkyl radical, m is ≧1, and wherein the fuel additive is essentially free of toxic constituents, to a diesel fuel/alkanol mixture to form a monophasic mixture.

15. A diesel fuel according to claim 4, wherein m is 4-16.

16. A diesel fuel according to claim 4, wherein m is 4-12.

17. A diesel fuel according to claim 10, wherein m is m=4 to m=8.
